(19) United States
(12) Patent Application Publication
(10) Pub. No.: US 2026/0283808 A1
(43) Pub. Date: Sep. 24, 2026

(54) KNEE PROSTHESIS WITH ANATOMIC GEOMETRY MOTION AND LAXITY

(71) Applicant: New York University, New York, NY (US)

(72) Inventor: Peter S. Walker, New York, NY (US)

(21) Appl. No.: 19/559,011

(22) Filed: Mar. 6, 2026

Related U.S. Application Data
(63) Continuation-in-part of application No. PCT/US2024/045822, filed on Sep. 9, 2024.
(60) Provisional application No. 63/581,188, filed on Sep. 7, 2023.
(60) Provisional application No. 63/933,680, filed on Dec. 8, 2025.

(51) Int. Cl. A61F 2/38; A61F 2/30

(52) U.S. Cl. CPC A61F 2/3859; A61F 2/389; A61F 2002/30131; A61F 2002/30237

(57) ABSTRACT

A knee prosthesis has a femoral component with a femoral bearing surface having a lateral section located adjacent in a medial-lateral direction to a central section that is adjacent in a medial-lateral direction to a medial section. The coronal and sagittal cross-sections of the lateral and medial sections are convex, and the central section has a central surface with a center of curvature eccentric to the lateral and medial sections in a sagittal plane. A tibial component has a tibial bearing surface in articulating contact with the femoral bearing surface, the tibial bearing surface having a lateral segment located adjacent in a medial-lateral direction to a central segment that is adjacent in a medial-lateral direction to a medial segment. A method for modifying laxity in a knee prosthesis is also described.

### CROSS-REFERENCE TO RELATED APPLICATIONS

This application is a continuation-in-part of International Application No. PCT/US2024/045822, filed Sep. 9, 2024, which claims priority to U.S. Provisional Patent Application No. 63/581,188, filed Sep. 7, 2023, and claims priority to U.S. Provisional Patent Application No. 63/933,680, filed Dec. 8, 2025, each of which is incorporated herein by reference in its entirety.

### BACKGROUND OF THE INVENTION

Several designs of artificial knee joints with different mechanical configurations have been described in literature. See for example U.S. Pat. Nos. 9,023,111 and 10,413,414, both to Walker. Several configurations are also available on the market. Most function reasonably well, however that is primarily because most patients who need an artificial knee do not frequently engage in strenuous or demanding activities. For the increasing number of patients who wish to return to an active lifestyle, there is evidence that the closer the mechanics of the artificial knee to normal, the better the patient is able to return to normal functions. Among designs today which typically provide the most anatomic mechanics are the medial pivot type and the medially congruent type.

There remains however a need in the art for an improved knee prosthesis that can perform better under strenuous or demanding activity. Embodiments of the knee prosthesis described herein fit this need.

### SUMMARY OF THE INVENTION

In one embodiment, a knee prosthesis includes a femoral component comprising a femoral bearing surface having a lateral section located adjacent in a medial-lateral direction to a central section that is adjacent in a medial-lateral direction to a medial section, where coronal and sagittal cross-sections of the lateral and medial sections are convex, and the central section has a central surface with a center of curvature eccentric to the lateral and medial sections in a sagittal plane; and a tibial component including a tibial bearing surface in articulating contact with the femoral bearing surface, the tibial bearing surface having a lateral segment located adjacent in a medial-lateral direction to a central segment that is adjacent in a medial-lateral direction to a medial segment, where the tibial bearing surface has an anterior-lateral quadrant having a first ratio of conformity, a posterior-lateral quadrant having a second ratio of conformity, an anterior-medial quadrant having a third ratio of conformity, and a posterior-medial quadrant having a fourth ratio of conformity. The ratio of conformity can be described as the ratio of radii of the femoral surface to the tibial surface in the sagittal plane. In one embodiment, the femoral component includes a medial distal-anterior having a steepened area in the sagittal view. In one embodiment, the tibial component includes a corresponding steepened area configured to increase resistance of anterior displacement on the tibial surface when an anterior shear force is applied. In one embodiment, the first ratio of conformity, second ratio of conformity, third ratio of conformity and fourth ratio of conformity each fall in a range between 20% and 90%. In one embodiment, the first ratio of conformity, second ratio of conformity, third ratio of conformity and fourth ratio of conformity are the same ratio. In one embodiment, the first ratio of conformity, second ratio of conformity, third ratio of conformity and fourth ratio of conformity are each a different ratio. In one embodiment, the central section of the femoral component forms a cylindrical convexity. In one embodiment, the central segment of the tibial component forms a cylindrical concavity. In one embodiment, the knee prosthesis includes a tibial insert defining the tibial bearing surface; wherein the tibial insert is configured to connect to the tibial component prior to implantation.

In one embodiment, a method for modifying laxity in a knee prosthesis includes the step of selecting each of the first ratio of conformity, second ratio of conformity, third ratio of conformity and fourth ratio of conformity based on a predetermined target laxity value. In one embodiment, the predetermined target laxity value comprises a rotational laxity value. In one embodiment, the predetermined target laxity value comprises a displacement laxity value. In one embodiment, a tibial insert defining the predetermined target laxity is connected to the tibial component prior to implantation.

In one embodiment, a knee prosthesis includes a femoral component having a femoral bearing surface having a lateral section located adjacent, in a medial-lateral direction to a central section that is adjacent in a medial-lateral direction to a medial section, wherein coronal and sagittal cross-sections of the lateral and medial sections are convex, and the central section has a central surface with a center of curvature eccentric to the lateral and medial sections in a sagittal plane; and a tibial component includes a tibial bearing surface in articulating contact with the femoral bearing surface, the tibial bearing surface having a lateral segment located adjacent, in a medial-lateral direction, to a central segment that is adjacent in a medial-lateral direction to a medial segment; wherein the femoral component includes a medial distal-anterior having a steepened area in the sagittal view and the tibial component having a corresponding steepened surface configured to increase resistance of anterior displacement on the tibial surface when an anterior shear force is applied. In one embodiment, the central section of the femoral component forms a cylindrical convexity. In one embodiment, the central segment of the tibial component forms a cylindrical concavity. In one embodiment, the knee prosthesis includes a tibial insert defining the tibial bearing surface; wherein the tibial insert is configured to connect to the tibial component prior to implantation.

In one embodiment, a knee prosthesis includes a femoral component comprising a femoral intercondylar bearing surface positioned between a lateral femoral condylar surface and a medial femoral condylar surface, and a tibial component having a tibial bearing surface in articulating contact with the femoral component, the tibial bearing surface comprising an anterior-lateral quadrant having a first ratio of conformity, a posterior-lateral quadrant having a second ratio of conformity, an anterior-medial quadrant having a third ratio of conformity, and a posterior-medial quadrant having a fourth ratio of conformity. In one embodiment, the femoral component includes a medial distal-anterior having a steepened area in the sagittal view. In one embodiment, the tibial component includes a corresponding steepened area configured to increase resistance of anterior displacement on the tibial surface when an anterior shear force is applied. In one embodiment, the first ratio of conformity, second ratio of conformity, third ratio of conformity and fourth ratio of conformity each fall in a range between 20% and 90%. In one embodiment, the first ratio of conformity, second ratio of conformity, third ratio of conformity and fourth ratio of conformity are the same ratio. In one embodiment, the first ratio of conformity, second ratio of conformity, third ratio of conformity and fourth ratio of conformity are each a different ratio. In one embodiment, the intercondylar bearing surface of the femoral component forms a cylindrical convexity. In one embodiment, a central segment of the tibial component forms a cylindrical concavity. In one embodiment, the knee prosthesis includes a tibial insert defining the tibial bearing surface; wherein the tibial insert is configured to connect to the tibial component prior to implantation.

In one embodiment, a knee prosthesis includes a femoral component comprising a femoral intercondylar bearing surface positioned between a lateral femoral condylar surface and a medial femoral condylar surface, and a tibial component having a tibial bearing surface in articulating contact with the femoral component, where the femoral component has a medial distal-anterior having a steepened area in the sagittal view and the tibial component has a corresponding steepened area configured to increase resistance of anterior displacement on the tibial surface when an anterior shear force is applied. In one embodiment, the tibial bearing surface includes an anterior-lateral quadrant having a first ratio of conformity, a posterior-lateral quadrant having a second ratio of conformity, an anterior-medial quadrant having a third ratio of conformity, and a posterior-medial quadrant having a fourth ratio of conformity. In one embodiment, the first ratio of conformity, second ratio of conformity, third ratio of conformity and fourth ratio of conformity each fall in a range between 20% and 90%. In one embodiment, the first ratio of conformity, second ratio of conformity, third ratio of conformity and fourth ratio of conformity are the same ratio. In one embodiment, the first ratio of conformity, second ratio of conformity, third ratio of conformity and fourth ratio of conformity are each a different ratio. In one embodiment, the femoral intercondylar bearing surface of the femoral component forms a cylindrical convexity. In one embodiment, a central segment of the tibial component forms a cylindrical concavity. In one embodiment, the knee prosthesis includes a tibial insert defining the tibial bearing surface; wherein the tibial insert is configured to connect to the tibial component prior to implantation.

In one embodiment, a knee prosthesis includes a femoral component comprising a femoral bearing surface having a lateral section located adjacent in a medial-lateral direction to a central section that is adjacent in a medial-lateral direction to a medial section, where coronal and sagittal cross-sections of the lateral and medial sections are convex, and the central section has a central surface with a center of curvature eccentric to the lateral and medial sections in a sagittal plane; and a tibial component including a tibial bearing surface in articulating contact with the femoral bearing surface, the tibial bearing surface having a lateral segment located adjacent in a medial-lateral direction to a central segment that is adjacent in a medial-lateral direction to a medial segment, where the tibial bearing surface has an anterior-lateral quadrant having a first ratio of conformity, a posterior-lateral quadrant having a second ratio of conformity, an anterior-medial quadrant having a third ratio of conformity, and a posterior-medial quadrant having a fourth ratio of conformity. The ratio of conformity can be described as the ratio of radii of the femoral surface to the tibial surface in the sagittal plane. In one embodiment, the femoral component includes a medial distal-anterior having a steepened area in the sagittal view. In one embodiment, the tibial component includes a corresponding steepened area configured to increase resistance of anterior displacement of the femoral surface on the tibial surface when an anterior shear force is applied. In one embodiment, the first ratio of conformity, second ratio of conformity, third ratio of conformity and fourth ratio of conformity each fall in a range between 20% and 90%. In one embodiment, the first ratio of conformity, second ratio of conformity, third ratio of conformity and fourth ratio of conformity are the same ratio. In one embodiment, the first ratio of conformity, second ratio of conformity, third ratio of conformity and fourth ratio of conformity are each a different ratio. In one embodiment, the central section of the femoral component forms a cylindrical convexity. In one embodiment, the central segment of the tibial component forms a cylindrical concavity. In one embodiment, the knee prosthesis includes a tibial insert defining the tibial bearing surface; wherein the tibial insert is configured to connect to the tibial baseplate component prior to implantation.

In one embodiment, a method for modifying laxity in a knee prosthesis includes the step of selecting each of the first ratio of conformity, second ratio of conformity, third ratio of conformity and fourth ratio of conformity based on a predetermined target laxity value. In one embodiment, the predetermined target laxity value comprises a rotational laxity value. In one embodiment, the predetermined target laxity value comprises a displacement laxity value. In one embodiment, a tibial insert defining the predetermined target laxity is connected to the tibial baseplate component prior to implantation.

In one embodiment, a knee prosthesis includes a femoral component having a femoral bearing surface having a lateral section located adjacent, in a medial-lateral direction to a central section that is adjacent in a medial-lateral direction to a medial section, wherein coronal and sagittal cross-sections of the lateral and medial sections are convex, and the central section has a central surface with a center of curvature eccentric to the lateral and medial sections in a sagittal plane; and a tibial component includes a tibial bearing surface in articulating contact with the femoral bearing surface, the tibial bearing surface having a lateral segment located adjacent, in a medial-lateral direction, to a central segment that is adjacent in a medial-lateral direction to a medial segment; wherein the femoral component includes a medial distal-anterior having a steepened area in the sagittal view and the tibial component having a corresponding steepened surface configured to increase resistance of anterior displacement of the femoral surface on the tibial surface when an anterior shear force is applied. In one embodiment, the central section of the femoral component forms a cylindrical convexity. In one embodiment, the central segment of the tibial component forms a cylindrical concavity. In one embodiment, the knee prosthesis includes a tibial insert defining the tibial bearing surface; wherein the tibial insert is configured to connect to the tibial baseplate component prior to implantation.

In one embodiment, a knee prosthesis includes a femoral component comprising a femoral intercondylar bearing surface positioned between a lateral femoral condylar surface and a medial femoral condylar surface, and a tibial component having a tibial bearing surface in articulating contact with the femoral component, the tibial bearing surface comprising an anterior-lateral quadrant having a first ratio of conformity, a posterior-lateral quadrant having a second ratio of conformity, an anterior-medial quadrant having a third ratio of conformity, and a posterior-medial quadrant having a fourth ratio of conformity. In one embodiment, the femoral component includes a medial distal-anterior having a steepened area in the sagittal view. In one embodiment, the tibial component includes a corresponding steepened area configured to increase resistance of anterior displacement of the femoral surface on the tibial surface when an anterior shear force is applied. In one embodiment, the first ratio of conformity, second ratio of conformity, third ratio of conformity and fourth ratio of conformity each fall in a range between 20% and 90%. In one embodiment, the first ratio of conformity, second ratio of conformity, third ratio of conformity and fourth ratio of conformity are the same ratio. In one embodiment, the first ratio of conformity, second ratio of conformity, third ratio of conformity and fourth ratio of conformity are each a different ratio. In one embodiment, the intercondylar bearing surface of the femoral component forms a cylindrical convexity. In one embodiment, a central segment of the tibial component forms a cylindrical concavity. In one embodiment, the knee prosthesis includes a tibial insert defining the tibial bearing surface; wherein the tibial insert is configured to connect to the tibial baseplate component prior to implantation.

In one embodiment, a knee prosthesis includes a femoral component comprising a femoral intercondylar bearing surface positioned between a lateral femoral condylar surface and a medial femoral condylar surface, and a tibial component having a tibial bearing surface in articulating contact with the femoral component, where the femoral component has a medial distal-anterior having a steepened area in the sagittal view and the tibial component has a corresponding steepened area configured to increase resistance of anterior displacement of the femoral surface on the tibial surface when an anterior shear force is applied. In one embodiment, the tibial bearing surface includes an anterior-lateral quadrant having a first ratio of conformity, a posterior-lateral quadrant having a second ratio of conformity, an anterior-medial quadrant having a third ratio of conformity, and a posterior-medial quadrant having a fourth ratio of conformity. In one embodiment, the first ratio of conformity, second ratio of conformity, third ratio of conformity and fourth ratio of conformity each fall in a range between 20% and 90%. In one embodiment, the first ratio of conformity, second ratio of conformity, third ratio of conformity and fourth ratio of conformity are the same ratio. In one embodiment, the first ratio of conformity, second ratio of conformity, third ratio of conformity and fourth ratio of conformity are each a different ratio. In one embodiment, the femoral intercondylar bearing surface of the femoral component forms a cylindrical convexity. In one embodiment, a central segment of the tibial component forms a cylindrical concavity. In one embodiment, the knee prosthesis includes a tibial insert defining the tibial bearing surface; wherein the tibial insert is configured to connect to the tibial baseplate component prior to implantation.

In one embodiment, a knee prosthesis includes a femoral component comprising a femoral bearing surface having a lateral section located adjacent in a medial-lateral direction to a central section that is adjacent in a medial-lateral direction to a medial section, wherein coronal and sagittal cross-sections of the lateral and medial sections are convex, the central section has a smooth convexity and a central surface with a center of curvature eccentric to the lateral and medial sections in a sagittal plane; and a tibial component comprising a tibial bearing surface in articulating contact with the femoral bearing surface, the tibial bearing surface having a lateral segment located adjacent in a medial-lateral direction to a central segment that is adjacent in a medial-lateral direction to a medial segment; wherein the tibial bearing surface comprises an anterior-lateral quadrant having a first ratio of conformity, a posterior-lateral quadrant having a second ratio of conformity, an anterior-medial quadrant having a third ratio of conformity, and a posterior-medial quadrant having a fourth ratio of conformity.

In one embodiment, the femoral component comprises a medial distal-anterior having a steepened area in the sagittal view. In one embodiment, the tibial component comprises a corresponding steepened area configured to increase resistance of anterior displacement on the tibial surface when an anterior shear force is applied. In one embodiment, the first ratio of conformity, second ratio of conformity, third ratio of conformity and fourth ratio of conformity each fall in a range between 20% and 90%. In one embodiment, the first ratio of conformity, second ratio of conformity, third ratio of conformity and fourth ratio of conformity are the same ratio. In one embodiment, the first ratio of conformity, second ratio of conformity, third ratio of conformity and fourth ratio of conformity are each a different ratio. In one embodiment, the central segment of the tibial component forms a smooth convexity. In one embodiment, the central segment forms a fifth ratio of conformity. In one embodiment, the knee prosthesis includes a tibial insert defining the tibial bearing surface; wherein the tibial insert is configured to connect to the tibial baseplate component prior to implantation.

In one embodiment, a method for modifying laxity in a knee prosthesis includes the steps of selecting each of the first ratio of conformity, second ratio of conformity, third ratio of conformity and fourth ratio of conformity based on a predetermined target laxity value. In one embodiment, the predetermined target laxity value comprises a rotational laxity value. In one embodiment, the predetermined target laxity value comprises a displacement laxity value. In one embodiment, a tibial insert defining the predetermined target laxity is connected to the tibial baseplate component prior to implantation.

In one embodiment, a knee prosthesis includes a femoral component comprising a femoral bearing surface having a lateral section located adjacent, in a medial-lateral direction to a central section that is adjacent in a medial-lateral direction to a medial section, wherein coronal and sagittal cross-sections of the lateral and medial sections are convex, and the central section has a smooth convexity and a central surface with a center of curvature eccentric to the lateral and medial sections in a sagittal plane; and a tibial component comprising a tibial bearing surface in articulating contact with the femoral bearing surface, the tibial bearing surface having a lateral segment located adjacent, in a medial-lateral direction, to a central segment that is adjacent in a medial-lateral direction to a medial segment; wherein the femoral component comprises a medial distal-anterior having a steepened area in the sagittal view and the tibial component comprises a corresponding steepened surface configured to increase resistance of anterior displacement of the femoral surface on the tibial surface when an anterior shear force is applied. In one embodiment, the central segment of the tibial component forms a smooth concavity. In one embodiment, the central segment forms a fifth ratio of conformity. In one embodiment, the knee prosthesis includes a tibial insert defining the tibial bearing surface; wherein the tibial insert is configured to connect to the tibial baseplate component prior to implantation.

In one embodiment, a knee prosthesis includes a femoral component comprising a femoral intercondylar bearing surface having a smooth convexity positioned between a lateral femoral condylar surface and a medial femoral condylar surface; and a tibial component having a tibial bearing surface in articulating contact with the femoral component, the tibial bearing surface comprising an anterior-lateral quadrant having a first ratio of conformity, a posterior-lateral quadrant having a second ratio of conformity, an anterior-medial quadrant having a third ratio of conformity, and a posterior-medial quadrant having a fourth ratio of conformity. In one embodiment, the femoral component comprises a medial distal-anterior having a steepened area in the sagittal view. In one embodiment, the tibial component comprises a corresponding steepened area configured to increase resistance of anterior displacement on the tibial surface when an anterior shear force is applied. In one embodiment, the first ratio of conformity, second ratio of conformity, third ratio of conformity and fourth ratio of conformity each fall in a range between 20% and 90%. In one embodiment, the first ratio of conformity, second ratio of conformity, third ratio of conformity and fourth ratio of conformity are the same ratio. In one embodiment, the first ratio of conformity, second ratio of conformity, third ratio of conformity and fourth ratio of conformity are each a different ratio. In one embodiment, a central segment of the tibial component forms a smooth concavity. In one embodiment, a central segment forms a fifth ratio of conformity. In one embodiment, the knee prosthesis includes a tibial insert defining the tibial bearing surface; wherein the tibial insert is configured to connect to the tibial baseplate component prior to implantation.

In one embodiment, a knee prosthesis includes a femoral component comprising a femoral intercondylar bearing surface having a smooth convexity positioned between a lateral femoral condylar surface and a medial femoral condylar surface; and a tibial component having a tibial bearing surface in articulating contact with the femoral component; wherein the femoral component comprises a medial distal-anterior having a steepened area in the sagittal view and the tibial component has a corresponding steepened area configured to increase resistance of anterior displacement of the femoral surface on the tibial surface when an anterior shear force is applied. In one embodiment, the tibial bearing surface comprises an anterior-lateral quadrant having a first ratio of conformity, a posterior-lateral quadrant having a second ratio of conformity, an anterior-medial quadrant having a third ratio of conformity, and a posterior-medial quadrant having a fourth ratio of conformity. In one embodiment, the first ratio of conformity, second ratio of conformity, third ratio of conformity and fourth ratio of conformity each fall in a range between 20% and 90%. In one embodiment, the first ratio of conformity, second ratio of conformity, third ratio of conformity and fourth ratio of conformity are the same ratio. In one embodiment, the first ratio of conformity, second ratio of conformity, third ratio of conformity and fourth ratio of conformity are each a different ratio. In one embodiment, a central segment of the tibial component forms a smooth concavity. In one embodiment, a central segment forms a fifth ratio of conformity. In one embodiment, the femoral and tibial bearing surfaces are articulated together at a 0 degree flexion angle. In one embodiment, the knee prosthesis includes a tibial insert defining the tibial bearing surface; wherein the tibial insert is configured to connect to the tibial baseplate component prior to implantation.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIGS. 1A-1C show views of a femoral component and illustrate formulation of an anatomically-based femoral component according to one embodiment. FIG. 1A shows perspective view of femoral component (top) and a tibial component (bottom) of a based on average knee from 100 mild arthritic MRIs, FIG. 1B shows cross-sectional views through lateral and medial condyles, and FIG. 1C shows a comparison between an average femur (top-left), and anatomic femoral component (top-right) a femoral component based on lateral condyle (bottom-left) and a femoral component with the dashed region pushed in (bottom-right).

FIGS. 2A-2C show views of a tibial component and illustrate definition of radii of a tibial insert according to one embodiment. FIG. 2A is a perspective view of a tibial insert having four sagittal tibial radii, and FIG. 2B and FIG. 2C are a cross-sectional view of a femoral component and a tibial component having customized reductions in anterior and posterior congruencies.

FIG. 3 is a perspective view of a femoral component having a steep anterior and tibial component having variable percentages of conformity in medial-posterior conformity, lateral-posterior conformity, medial-anterior conformity and lateral-anterior conformity according to one embodiment.

FIGS. 4A and 4B show views illustrating the reproduction of lateral femoral rollback according to one embodiment. FIG. 4A is a perspective view of a femoral component and a tibial component with illustrated sections through the femur and tibia, and FIG. 4B shows degrees of flexion at 0, 30, 60, 90 and 120 degrees in each of a composite view, central section, medial section and lateral section.

FIG. 5 is a perspective view of a femoral component central section having a smooth convexity and a corresponding tibial component with a central concavity according to one embodiment.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in knee prosthesis systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“Approximately” as used herein when referring to a measurable value such as an amount, a temporal duration, and the like, is meant to encompass variations from the specified value, as such variations are appropriate.

“Femcon” as used herein means femoral component.

Referring now in detail to the drawings, in which like reference numerals indicate like parts or elements throughout the several views, in various embodiments, presented herein is a knee prosthesis system and method with anatomic geometry motion and laxity.

Embodiments of the knee prosthesis described herein include several unique features, including (a) component geometries based on an average three-dimensional anatomic shape of knees, (b) bearing surface shapes which will allow three-dimensional anatomic motions to occur in a range of activities, (c) bearing surfaces which control the values of laxities including displacements and rotations, (d) tibial bearing surfaces which are tuned to accomplish the motion and laxity requirements separately for each quadrant (anterior-lateral, posterior-lateral, anterior-medial and posterior-medial), (e) tuning involving a percentage reduction in height of the tibial curvatures from fully conforming with the femoral surfaces, to values which will provide the requisite laxity characteristics, and (f) intercondylar bearing surfaces which will guide the femoral component to rotate externally as the knee is flexed, while maintaining a relatively fixed position of the medial condyles, combined with posterior displacement of the lateral femoral condyle, thus replicating normal anatomic motion behavior.

Embodiments of the knee prosthesis can be manufactured from materials such as cobalt-chrome alloy, titanium alloy, and ulra-high molecular weight polyethylene. The femoral component can be a close match in shape for the anatomic knee which it is replacing. This will minimize overhang at the sides which would irritate collateral ligaments and capsular tissue, while minimizing under-hang which will leave exposed areas of uncovered bone, subject to bleeding, and reduce the varus-valgus stability. The same applies to the peripheral shape and dimensions of the tibial component. Bearing surfaces are designed to respond anatomically to the forces and moments imposed during activities. The main four bearing surfaces of the tibial surface, with their own individual ratio of conformity with the femoral component, provide precise control of laxity. In other words, the displacements and rotations will be similar to anatomic, giving the patient a normal feeling to the knee function.

In particular, excess laxity will be felt as an unstable knee. Inadequate laxity will results in a feeling of restriction, and will also impose high shear and torque forces to the components, increasing wear, and possibly causing loosening. Embodiments described herein allow for optimizing laxity in a knee prosthesis. The higher range of flexion facilitates activities such as climbing steep stairs, rising from a low chair, getting in and out of restricted spaces such as automobiles.

For reproducing anatomic form and function, embodiment of an artificial knee have one or more of the following characteristics:
(1) An overall femoral component shape which is based on the average 3-dimensional femoral shape from a large collection of arthritic knees. On the medial distal-anterior region, a steepened area seen in the sagittal view, to increase the resistance of anterior displacement on the tibial surface, when an anterior shear force is applied.
(2) Tibial bearing surfaces on the lateral and medial sides which are generated by initially being perfectly conforming to the femoral surfaces, but then are reduced in steepness in four quadrants (ant-med, post-med, ant-lat, post-lat) by a ratio such as to produce anatomic laxity values. On the lateral side the steepness is reduced the most, ant-med the least.
(3) Intercondylar bearing surfaces to generate lateral femoral rollback and a stable medial side during flexion, which is anatomic behavior. This can be achieved by a cylindrical-like concavity in the center of the tibia, interacting with a convex cylindrical-like surface on the femoral component.

The embodiments described in more detail below may include a standard femoral bearing surface with a tibial surface such that there are 4 different conformity ratios. The range of conformities may be specified. In one embodiment, the range of conformity is a high conformity medially and low conformity laterally. A distal anterior medial area may include femoral and tibial surfaces that are steepened. In one embodiment, the femoral component includes a central femoral intercondylar bearing articulating in a central tibial intercondylar bearing. On the femoral component, in the sagittal view, the center of curvature of the intercondylar surface is eccentric to the lateral and medial femoral condyles. In the frontal view, the intercondylar surface can be cylindrical, or be concave or convex. In one embodiment, the intercondylar surface on the femoral component forms a cylindrical convexity. The interaction between the femoral and tibial intercondylar bearing surfaces guides the femur to rotate externally in extension (femoral contact point moves anteriorly on the lateral side) and rotate internally in flexion (femoral contact point moves posteriorly on the lateral side). The high medial conformity and low lateral conformity on the medial and lateral main bearing surfaces, causes small anterior-posterior displacements medially, and large anterior-posterior displacements laterally.

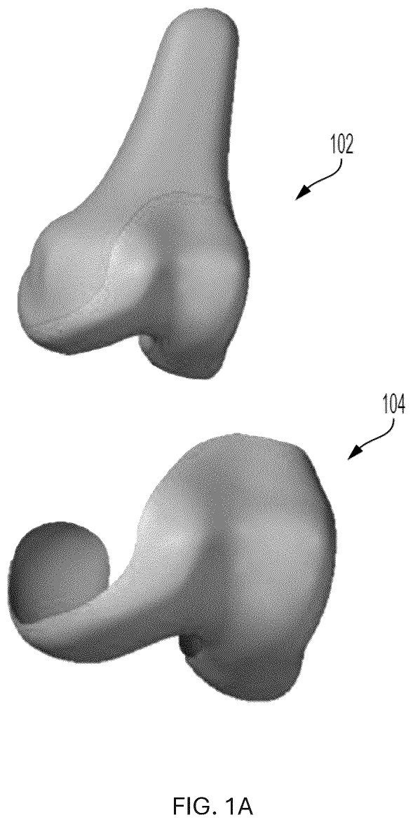

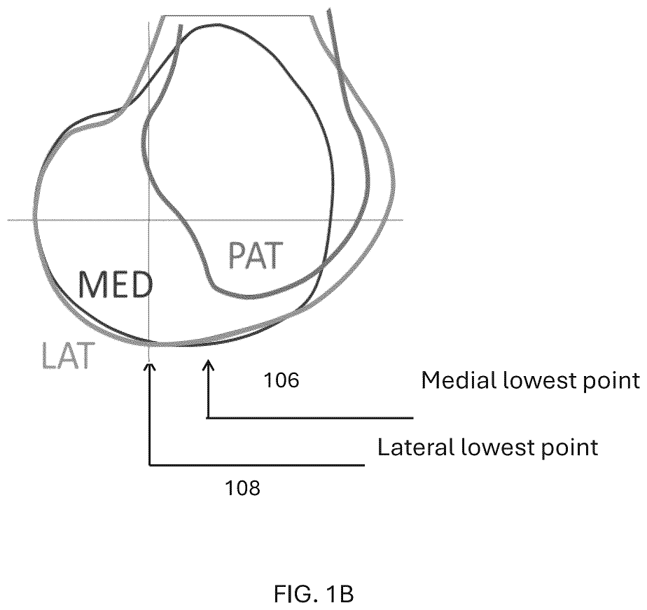

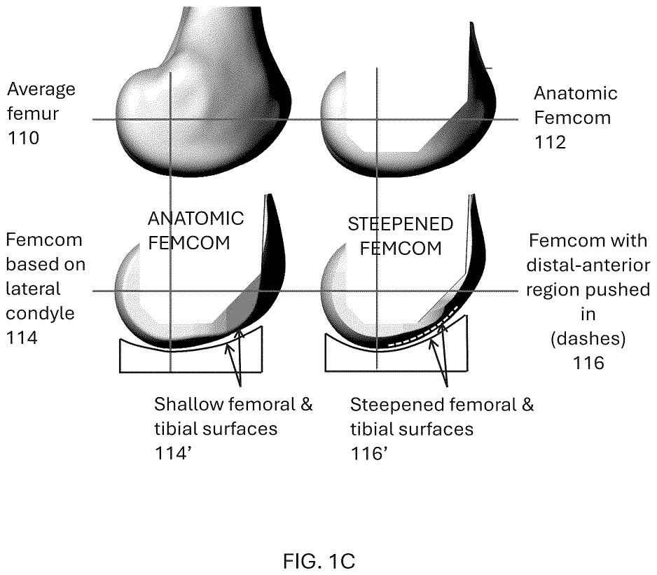

With reference now to FIG. 1A, a femoral component 102 and tibial component 104 is shown based on an average knee from 100 mild arthritic MRIs is show in one embodiment. This data can be retrieved from a database such as the National Arthritis Database. With reference to FIG. 1B, cross-sectional views through lateral and medial condyles are shown in one embodiment, with the medial lowest point 106 and the lateral lowest point 108 identified. FIG. 1C shows in one embodiment a comparison between an average femur 110, and anatomic femoral component 112, a femoral component based on lateral condyle 114 and a femoral component with the dashed region pushed in 116. The femoral component based on lateral condyle 114 features shallow femoral and tibial bearing surfaces 114′. The femoral component with the dashed region pushed in 116 features steepened femoral and tibial bearing surfaces 116′. Thus the femoral component in one embodiment has a medial distal-anterior having a steepened area in the sagittal view. The tibial component may have a corresponding steepened area to increase resistance of anterior displacement on the tibial surface when an anterior shear force is applied.

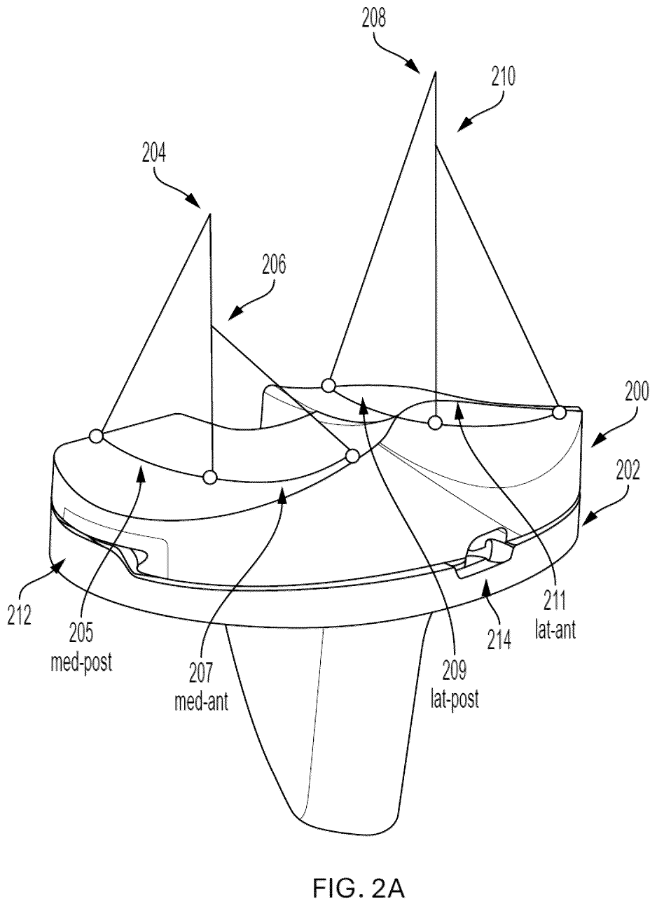

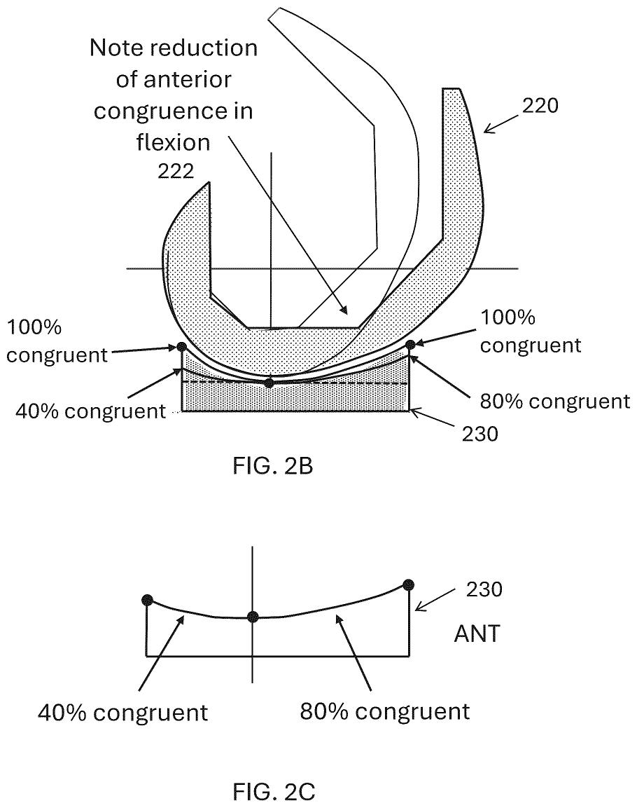

With reference now to FIG. 2A a tibial component illustrate radii of a tibial insert is shown in one embodiment. The tibial insert 200 can connect to a baseplate 202 using connection methods known in the art, such as for example screws, adhesives, clips, snap-fits, friction-fits, mating sections or interlocking mechanisms. The base 202 and tibial insert 200 may for example include a series of structures 212, 214 for proper alignment (e.g. tongue and groove portions, protrusions and recesses, or similar structural features at the mating surfaces). The tibial insert 200 has four sagittal tibial radii 204, 206, 208 and 210. Depending on the ratio of conformity for each of the medial-posterior 205, medial-anterior 207, lateral-posterior 209 and lateral anterior 211 sections of the tibial bearing surface, the position of the sagittal tibial radii 204, 206, 208 and 210 can be modified. The ratio of conformity can be described as the ratio of radii of the femoral surface to the tibial surface in the sagittal plane.

FIG. 2B and FIG. 2C show in one embodiment a cross-sectional view of a femoral component 220 and a tibial 230 component having customized reductions in anterior and posterior congruencies. A reduction of anterior congruence in flexion 222 is highlighted. In this embodiment, the tibial component 230 is modified to have a 40% posterior congruence and an 80% anterior congruence with the femoral component 220. Other congruencies can be implemented based on for example the patent anatomy and characteristics and the goals for patient movement.

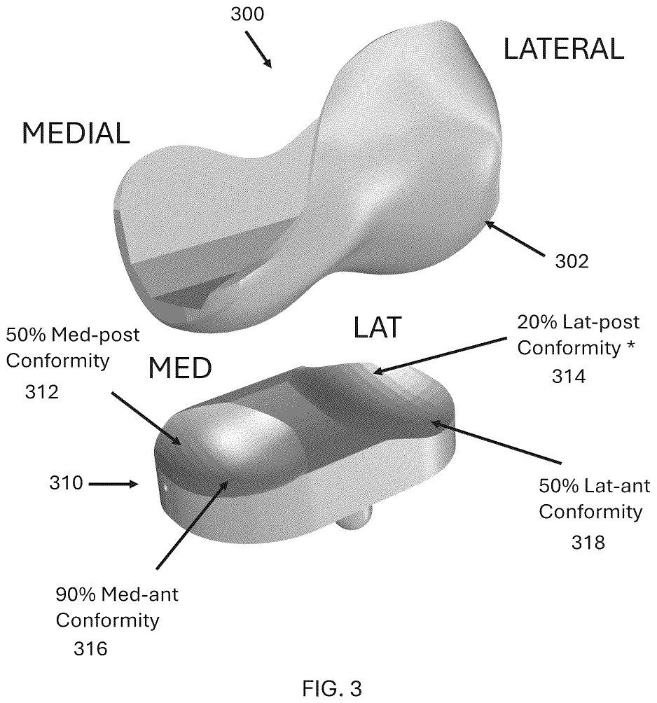

With reference now to FIG. 3, bearing surfaces based on matching anatomic laxities are show according to one embodiment. A femoral component 300 has a steep anterior 302 and tibial component 310 has variable percentages of conformity, in this embodiment in 50% medial-posterior conformity 312, 20% lateral-posterior conformity 314, 90% medial-anterior conformity 316 and 50% lateral-anterior conformity 318.

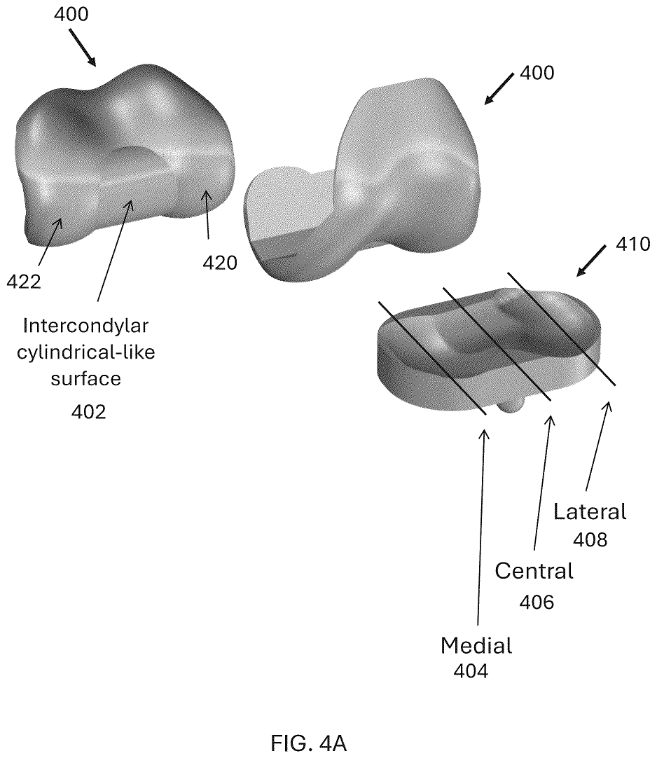

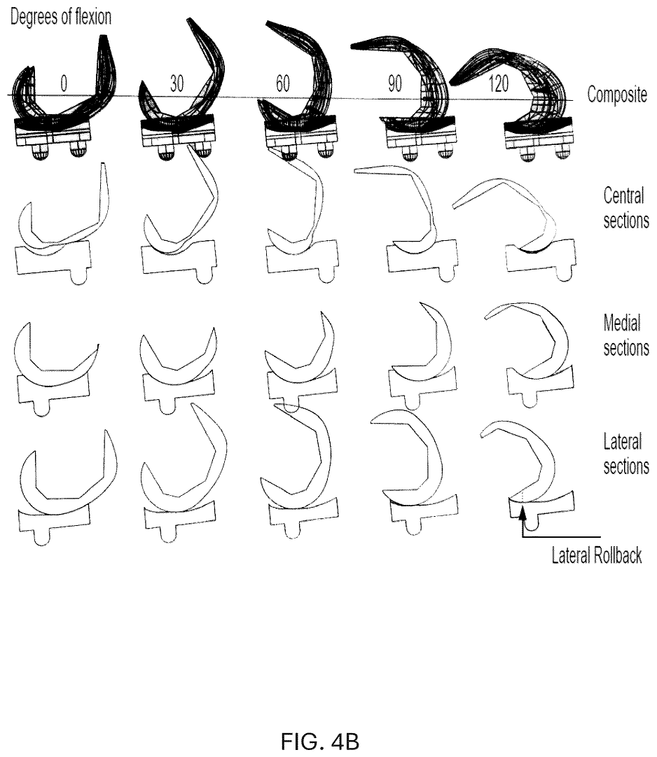

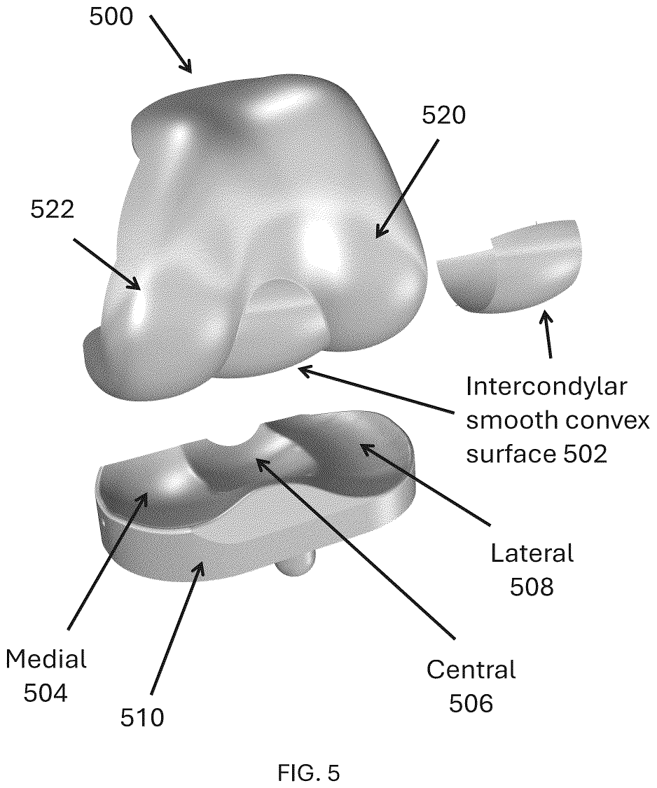

With reference now to FIG. 4A, illustrating the reproduction of lateral femoral rollback according to one embodiment, a femoral component 400 and a tibial component 410 are shown with illustrated medial 404, central 406 and lateral 408 sections through the femur and tibia. The intercondylar cylindrical-like surface 402 has a center which is eccentric to the centers of the lateral 420 and medial 422 femoral condyles. The purpose of the eccentricity is so that in flexion-extension, the femoral intercondylar surfaces interacting with the tibial central intercondylar surface, causes the femur to displace posterior on the tibia in flexion and to displace anterior on the tibia in extension. It is noted that because the medial bearing surfaces of the femur and tibia are in relatively closer conformity, compared with less conformity on the lateral side, the anterior-posterior displacements are larger on the lateral side than on the medial side. This pattern of motion resembles that of the normal anatomic knee. Thus, the central section of the femoral component may form a cylindrical convexity while the central segment of the tibial component forms a cylindrical concavity. FIG. 4B shows degrees of flexion at 0, 30, 60, 90 and 120 degrees in each of a composite view, central section, medial section and lateral section.

With reference now to FIG. 5, in one embodiment, a femoral component 500 and a tibial component 510 are shown. In this embodiment, the intercondylar surface 502 has a smooth convex shape, also with a center in the sagittal plane that is eccentric to the centers of the lateral 520 and medial 522 femoral condyles. In the frontal view, the femoral component 500 has an intercondylar surface 502 that may be considered to have a smooth convexity, while the central segment of the tibial surface 506 forms an a smooth concavity between medial 504 and lateral 508 surfaces. The smooth convexity may include for example a smoothly rounded continuous convex curve in the frontal view. It may for example be a symmetrical curve such as a curve on an oval, or an asymmetrical curve such as an ovoid-like curve that is broader at the bottom and gradually tapers towards the top (e.g. an egg-like profile). Thus embodiments may feature symmetrical convex curves that are circular, oval or otherwise closer to symmetrical than an asymmetrical ovoid. An ellipse or elliptical curve may in some embodiments be implemented, closely or perfectly balanced around two axes, creating a uniform, symmetrical outline. Ellipsoids and spheroids may extend these smooth, convex curves into three dimensions, producing evenly rounded volumes. More generally, the smooth convexity can be any smooth convex curve. A smooth convexity at the intercondylar surface 502 allows for preservation of more surface area of the lateral 520 and medial 522 condyles. This provides an advantage because the condyles 520, 522 are the main load-bearing elements whereas the smooth convex surface 502 is motion-guiding with smaller loads.

In one embodiment, a method for modifying laxity in a knee prosthesis includes selecting each of the first ratio of conformity, second ratio of conformity, third ratio of conformity and fourth ratio of conformity based on a predetermined target laxity value. In one embodiment, the predetermined target laxity value comprises a rotational laxity value. In one embodiment, the predetermined target laxity value comprises a displacement laxity value. In one embodiment, a tibial insert defining the predetermined target laxity is connected to the tibial baseplate component prior to implantation.

Components of the artificial knee system may include a femoral component and a choice of for example two to four tibial plastic tibial components which have different laxities, from constrained to unconstrained. From an examination of the patient pre-operative or during the operation, the surgeon can designate the level of laxity required for that patient. The surgeon may then select the appropriate plastic tibial insert. Each tibial insert may correspond to four different radii which define the total surface. The set of tibial insert designs, each comprising four different radii in the four quadrants which collectively determine the laxity of the knee, each have a different laxity according to the anatomic spectrum, whereby the surgeon selects the appropriate insert based on a laxity examination of the patient.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

1. A knee prosthesis comprising:
a femoral component comprising a femoral bearing surface having a lateral section located adjacent in a medial-lateral direction to a central section that is adjacent in a medial-lateral direction to a medial section, wherein coronal and sagittal cross-sections of the lateral and medial sections are convex, and the central section has a central surface with a center of curvature eccentric to the lateral and medial sections in a sagittal plane; and
a tibial component comprising a tibial bearing surface in articulating contact with the femoral bearing surface, the tibial bearing surface having a lateral segment located adjacent in a medial-lateral direction to a central segment that is adjacent in a medial-lateral direction to a medial segment;
wherein the tibial bearing surface comprises an anterior-lateral quadrant having a first ratio of conformity, a posterior-lateral quadrant having a second ratio of conformity, an anterior-medial quadrant having a third ratio of conformity, and a posterior-medial quadrant having a fourth ratio of conformity.

2. The knee prosthesis of claim 1, wherein the femoral component comprises a medial distal-anterior having a steepened area in the sagittal view.

3. The knee prosthesis of claim 2, wherein the tibial component comprises a corresponding steepened area configured to increase resistance of anterior displacement on the tibial surface when an anterior shear force is applied.

4. The knee prosthesis of claim 1, wherein the first ratio of conformity, second ratio of conformity, third ratio of conformity and fourth ratio of conformity each fall in a range between 20% and 90%.

5. The knee prosthesis of claim 1, wherein the first ratio of conformity, second ratio of conformity, third ratio of conformity and fourth ratio of conformity are the same ratio.

6. The knee prosthesis of claim 1, wherein the first ratio of conformity, second ratio of conformity, third ratio of conformity and fourth ratio of conformity are each a different ratio.

7. The knee prosthesis of claim 1, wherein the central section of the femoral component forms a cylindrical convexity.

8. The knee prosthesis of claim 7, wherein the central segment of the tibial component forms a cylindrical concavity.

9. The knee prosthesis of claim 1 further comprising:
a tibial insert defining the tibial bearing surface;
wherein the tibial insert is configured to connect to the tibial component prior to implantation.

10-13. (canceled)

14. A knee prosthesis comprising:
a femoral component comprising a femoral bearing surface having a lateral section located adjacent, in a medial-lateral direction to a central section that is adjacent in a medial-lateral direction to a medial section, wherein coronal and sagittal cross-sections of the lateral and medial sections are convex, and the central section has a central surface with a center of curvature eccentric to the lateral and medial sections in a sagittal plane; and
a tibial component comprising a tibial bearing surface in articulating contact with the femoral bearing surface, the tibial bearing surface having a lateral segment located adjacent, in a medial-lateral direction, to a central segment that is adjacent in a medial-lateral direction to a medial segment;
wherein the femoral component comprises a medial distal-anterior having a steepened area in the sagittal view and the tibial component comprises a corresponding steepened surface configured to increase resistance of anterior displacement on the tibial surface when an anterior shear force is applied.

15. The knee prosthesis of claim 14, wherein the central section of the femoral component forms a cylindrical convexity.

16. The knee prosthesis of claim 15, wherein the central segment of the tibial component forms a cylindrical concavity.

17. The knee prosthesis of claim 14 further comprising:
a tibial insert defining the tibial bearing surface;
wherein the tibial insert is configured to connect to the tibial component prior to implantation.

18-35. (canceled)

36. A knee prosthesis comprising:
a femoral component comprising a femoral bearing surface having a lateral section located adjacent in a medial-lateral direction to a central section that is adjacent in a medial-lateral direction to a medial section, wherein coronal and sagittal cross-sections of the lateral and medial sections are convex, the central section has a smooth convexity and a central surface with a center of curvature eccentric to the lateral and medial sections in a sagittal plane; and
a tibial component comprising a tibial bearing surface in articulating contact with the femoral bearing surface, the tibial bearing surface having a lateral segment located adjacent in a medial-lateral direction to a central segment that is adjacent in a medial-lateral direction to a medial segment;
wherein the tibial bearing surface comprises an anterior-lateral quadrant having a first ratio of conformity, a posterior-lateral quadrant having a second ratio of conformity, an anterior-medial quadrant having a third ratio of conformity, and a posterior-medial quadrant having a fourth ratio of conformity.

37. The knee prosthesis of claim 36, wherein the femoral component comprises a medial distal-anterior having a steepened area in the sagittal view.

38. The knee prosthesis of claim 37, wherein the tibial component comprises a corresponding steepened area configured to increase resistance of anterior displacement of the femoral surface on the tibial surface when an anterior shear force is applied.

39. The knee prosthesis of claim 36, wherein the first ratio of conformity, second ratio of conformity, third ratio of conformity and fourth ratio of conformity each fall in a range between 20% and 90%.

40. The knee prosthesis of claim 36, wherein the first ratio of conformity, second ratio of conformity, third ratio of conformity and fourth ratio of conformity are the same ratio.

41. The knee prosthesis of claim 36, wherein the first ratio of conformity, second ratio of conformity, third ratio of conformity and fourth ratio of conformity are each a different ratio.

42. The knee prosthesis of claim 36, wherein the central segment of the tibial component forms a smooth concavity.

43-66. (canceled)
